(19) United States
(12) Patent Application Publication
(10) Pub. No.: US 2013/0231744 A1
(43) Pub. Date: Sep. 5, 2013

(54) GROMMET FOR USE WITH SURGICAL IMPLANT

(71) Applicant: SOLANA SURGICAL, LLC, Memphis, TN (US)

(72) Inventors: Alan G. Taylor, Memphis, TN (US); Rebecca H. Wahl, Escondido, CA (US)

(73) Assignee: SOLANA SURGICAL, LLC, Memphis, TN (US)

(21) Appl. No.: 13/781,378

(22) Filed: Feb. 28, 2013

Related U.S. Application Data
(60) Provisional application No. 61/605,379, filed on Mar. 1, 2012.

(51) Int. Cl. A61F 2/28

(52) U.S. Cl. CPC A61F 2/28
USPC 623/16.11

(57) ABSTRACT

A grommet (414) for use with a surgical implant (212) comprises a base (450) and a sleeve (452). The base (450) can be positioned around a portion of the surgical implant (212). Additionally, the sleeve (452) is connected to the base (450) and extends away from the base (450). The sleeve (452) includes an inner surface (446) having a non-circular shape to inhibit relative rotation between the grommet (414) and the surgical implant (212), and an outer surface (438) that is substantially cylinder-shaped. The outer surface (438) of the sleeve (452) can taper as the sleeve (452) extends away from the base (450). Additionally, the sleeve (452) can include one or more anti-rotation features (440) that are positioned along the outer surface (438) of the sleeve (452) to inhibit rotation between the grommet (414) and the bone (20) into which the surgical implant (212) is implanted.

### RELATED INVENTION

This application claims priority on U.S. Provisional Application Ser. No. 61/605,379, filed Mar. 1, 2012 and entitled “GROMMET FOR USE WITH SURGICAL IMPLANT”. As far as permitted, the contents of U.S. Provisional Application Ser. No. 61/605,379 are incorporated herein by reference.

### BACKGROUND

It is well known that some people have problems with one or more joints in their body, including feet and/or hands. Treatment of such problems with the joints often entails to inserting of a surgical implant into the joint in order to stabilize the joint, while still allowing for good joint mobility, good joint load transfer and good joint purchase. Unfortunately, the treatment of such joint problems with existing surgical implants has not been entirely satisfactory.

### SUMMARY

The present invention is directed toward a grommet for use with a surgical implant. In certain embodiments, the grommet comprises a base and a sleeve. The base can be positioned around a portion of the surgical implant. Additionally, the sleeve is connected to the base and extends away from the base. The sleeve includes an inner surface having a non-circular shape, and an outer surface that is substantially cylinder-shaped.

In some embodiments, the outer surface of the sleeve tapers as the sleeve extends away from the base. For example, in such embodiments, the outer surface of the sleeve can have a taper angle of between approximately 20.0 and 25.0 degrees.

Additionally, in certain embodiments, the sleeve can include one or more anti-rotation features that are positioned along the outer surface of the sleeve. The anti-rotation features can be varied. For example, the anti-rotation features can include one or more anti-rotation ribs that extend away from the outer surface of the sleeve, and/or one or more anti-rotation grooves that are formed into the outer surface of the sleeve.

In one embodiment, the base is inhibited from rotating relative to the portion of the surgical implant.

Further, in some embodiments, the surgical implant can include a stem, and the grommet can be positioned around a portion of the stem. In certain such embodiments, the stem has an outer surface having a first shape, and the inner surface of the sleeve has a second shape, the second shape being substantially similar to the first shape. This enables the grommet to fit snugly and securely around the stem. In one embodiment, the second shape can be non-circular such that the grommet is inhibited from rotating relative to the stem.

Additionally, in one embodiment, the present invention is further directed toward a surgical implant for implanting into bone material, the surgical implant including a stem and the grommet as described above that is positioned around the stem. Moreover, in such embodiment, the sleeve can include one or more anti-rotation features that are positioned along the outer surface of the sleeve, the anti-rotation features inhibiting the grommet from rotating relative to the bone material.

In another application, the present invention is directed toward a grommet for use with a surgical implant that can be implanted into bone material, the grommet comprising (i) a base that can be positioned around a portion of the surgical implant; and (ii) a sleeve that is connected to the base and extends away from the base, the sleeve including an outer surface that is substantially cylinder-shaped, and one or more anti-rotation features that are positioned along the outer surface of the sleeve.

Further, in still another application, the present invention is directed toward a grommet for use with a surgical implant that can be implanted into bone material, the surgical implant including a stem, the grommet comprising (i) a base that can be positioned around a portion of the stem; and (ii) a sleeve that is connected to the base and extends away from the base, the sleeve including (i) an inner surface having a non-circular shape such that the grommet is inhibited from rotating relative to the stem, (ii) an outer surface that is substantially cylinder-shaped, and (iii) one or more anti-rotation features that are positioned along the outer surface of the sleeve, wherein when the surgical implant is implanted into the bone material, the one or more anti-rotation features inhibit the grommet from rotating relative to the bone material.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a side view of a part of a foot with a surgical implant implanted therein, the surgical implant including a pair of grommets having features of the present invention included therewith;

FIG. 1B is a side view of the part of the foot and the surgical implant having the grommets of FIG. 1A included therewith, with the foot in a flexed position;

FIG. 2A is a perspective view of an embodiment of a surgical implant including a pair of grommets having features of the present invention included therewith;

FIG. 2B is a side view of the surgical implant including the pair of grommets of FIG. 2A;

FIG. 2C is a top view of the surgical implant including the pair of grommets of FIG. 2A;

FIG. 2D is a sectional view of the surgical implant including the pair of grommets taken on line D-D in FIG. 2B;

FIG. 3 is a perspective view of another embodiment of a surgical implant including a pair of grommets having features of the present invention included therewith;

FIG. 4A is a perspective view of an embodiment of a grommet having features of the present invention;

FIG. 4B is an end view of the grommet of FIG. 4A;

FIG. 4C is a side view of the grommet of FIG. 4A;

FIG. 4D is a top view of the grommet of FIG. 4A;

FIG. 5A is a perspective view of another embodiment of a grommet having features of the present invention;

FIG. 5B is an end view of the grommet of FIG. 5A;

FIG. 5C is a side view of the grommet of FIG. 5A;

FIG. 5D is a sectional view of the grommet taken on line D-D in FIG. 5B;

FIG. 5E is another sectional view of the grommet taken on line E-E in FIG. 5B;

FIG. 6 is a simplified side view of an embodiment of a cannulated tapered drill bit that can be used with the present invention;

FIG. 7 is a simplified side view of another embodiment of a cannulated tapered drill bit that can be used with the present invention; and

FIG. 8 is a simplified side view of a wire that can be used with the drill bits of FIGS. 6 and 7.

### DESCRIPTION

The present invention is directed to a grommet for use with a surgical implant that can be used to treat joint problems in the feet and/or hands. More specifically, in certain embodiments, the surgical implant and one or more grommets can be effectively utilized in conjunction with one another to treat one or more joint problems in the feet or toes, e.g., a metatarsal phalangeal joint or an interphalangeal joint of one of the toes.

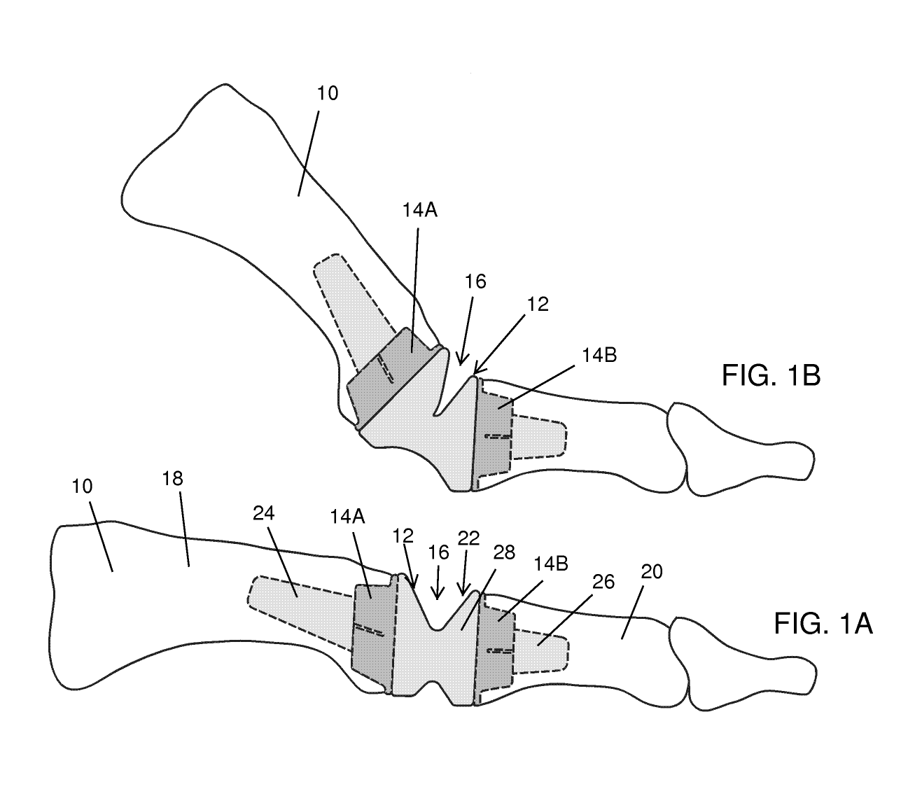

FIG. 1A is a side view of a part of a foot 10 with a surgical implant 12 implanted therein. As illustrated, the surgical implant 12 includes one or more grommets 14 (two are illustrated in FIG. 1A) having features of the present invention provided therewith. The grommets 14, as disclosed in detail herein, can be used with a variety of different surgical implants. For example, suitable non-exclusive surgical implants 12 for use with the present invention can be as disclosed in (i) U.S. Pat. No. 6,319,284 issued to Rushdy et al. and assigned to Futura Biomedical LLC, (ii) U.S. Pat. No. D490,900 issued to Ogilvie et al. and assigned to Ascension Orthopedics, Inc., (iii) U.S. Pat. No. 3,875,594 issued to Swanson and assigned to Dow Corning Corporation, and (iv) U.S. Pat. No. 6,869,449 issued to Ball et al. and assigned to DePuy Orthopaedics, Inc.

FIG. 1A illustrates one embodiment of the surgical implant 12 that may be used in conjunction with the grommets 14 that are disclosed in detail herein. As illustrated, the surgical implant 12 can be used to provide a replacement joint in the first metatarsal phalangeal joint 16 (sometimes referred to herein simply as the “joint”) between the metatarsal 18 and the proximal phalanx 20. Alternatively, the surgical implant 12 can be used in another suitable joint. In this embodiment, the surgical implant 12 includes an implant body 22 having a proximal stem 24 (illustrated in phantom), a distal stem 26 (illustrated in phantom) and a hinge 28.

In the embodiment of the surgical implant 12 illustrated in FIG. 1A, the proximal stem 24 is sized and shaped to be inserted into a medullary canal in the metatarsal 18. Somewhat similarly, the distal stem 26 is sized and shaped to be inserted into a medullary canal in the proximal phalanx 20. The hinge 28 is positioned substantially between and connects the proximal stem 24 and the distal stem 26. Additionally, the hinge 28 allows for relative movement, e.g., flexing, between the proximal stem 24 and the distal stem 26 during movement of the foot 10.

In certain embodiments, the one or more grommets 14 can include a first grommet 14A and a second grommet 14B. For example, as illustrated in FIG. 1A, the first grommet 14A can be positioned around a portion of the proximal stem 24 substantially adjacent to the hinge 28, and the second grommet 14B can be positioned around a portion of the distal stem 26 substantially adjacent to the hinge 28. Additionally and/or alternatively, one or both of the first grommet 14A and the second grommet 14B can be integrally formed with the surgical implant 12.

As an overview, the grommets 14A, 14B are uniquely designed to maintain the surgical implant 12 positioned appropriately within the joint, e.g., the first metatarsal phalangeal joint 16, while allowing certain relative movement between the stems, i.e. the proximal stem 24 and the distal stem 26, and the corresponding bones 18, 20, and between the stems 24, 26 and the corresponding grommets 14A, 14B. More specifically, in this embodiment, the first grommet 14A allows the proximal stem 24 to move relative to the metatarsal 18, i.e. within the medullary canal, and/or relative to the first grommet 14A, while maintaining the proper positioning of the proximal stem 24 within the first metatarsal phalangeal joint 16. Somewhat similarly, the second grommet 14B allows the distal stem 26 to move relative to the proximal phalanx 20, i.e. within the medullary canal, and/or relative to the second grommet 14B, while maintaining the proper positioning of the distal stem 26 within the first metatarsal phalangeal joint 16.

With this design, the grommets 14A, 14B can limit the direct interface between the surgical implant 12 and the bones 18, 20, thereby inhibiting discomfort experienced by the patient that may occur due to such direct interface. For example, the surgical implant 12 may be made of a silicone material, and relative movement between the surgical implant 12 and the bone material 18, 20 at an interface may result in certain debris being generated within the joint 16. The use of grommets 14A, 14B as described herein, can inhibit the generation of such debris within the joint 16. Additionally, the grommets 14A, 14B remain substantially stationary relative to the bones 18, 20, while ensuring that the surgical implant 12 remains properly positioned within the joint 16. Moreover, the grommets 14A, 14B are designed to allow for improved range of motion of the joint 16 after insertion of the surgical implant 12

FIG. 1B is a side view of the part of the foot 10 and the surgical implant 12 having the grommets 14A, 14B of FIG. 1A included therewith. Additionally, FIG. 1B illustrates the foot 10 in a flexed position. In particular, FIG. 1B demonstrates the ability of the foot 10 to properly flex with the surgical implant 12 having the grommets 14A, 14B included therewith inserted within the joint 16. As illustrated, during flexing of the foot 10, the grommets 14A, 14B assist in maintaining the surgical implant 12 properly positioned within the joint 16. Although the flexing of the foot 10 may involve some movement between the proximal stem 24 (illustrated in phantom) and the medullary canal in the metatarsal 18, and/or between the distal stem 26 (illustrated in phantom) and the medullary canal in the proximal phalanx 20, the grommets 14A, 14B remain substantially stationary relative to the bones 18, 20.

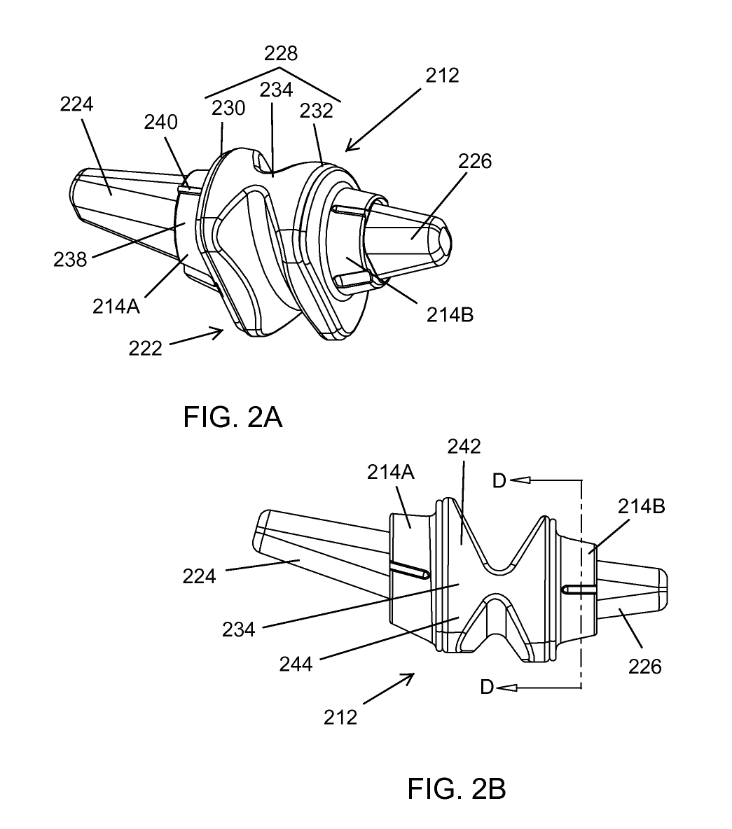

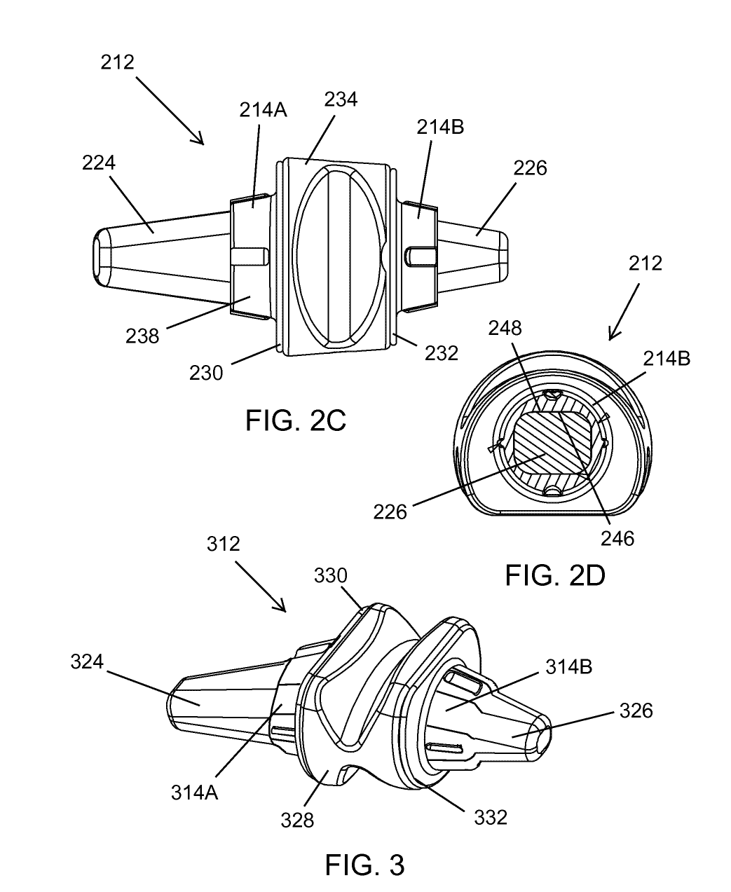

FIG. 2A is a perspective view of an embodiment of a surgical implant 212 including a pair of grommets 214A, 214B having features of the present invention included therewith. The design of the surgical implant 212 and the design of the grommets 214A, 214B can be varied according to the specific requirements for the surgical implant 212.

As illustrated in FIG. 2A, the surgical implant 212 includes an implant body 222 having a proximal stem 224, a distal stem 226, and a hinge 228.

As discussed above, the proximal stem 224 and the distal stem 226 are sized and shaped to fit within canals or holes in the bones on either side of a joint 16 (illustrated in FIG. 1A).

The hinge 228 is positioned substantially between and connects the proximal stem 224 and the distal stem 226. Additionally, the hinge 228 allows for relative movement, e.g., flexing, between the proximal stem 224 and the distal stem 226 during movement of the foot 10 (illustrated in FIG. 1A). As illustrated, the hinge 228 can include a proximal hinge buttress 230, a distal hinge buttress 232, and a hinge center section 234 that extends between and connects the proximal hinge buttress 230 and the distal hinge buttress 232. The hinge center section 234 is designed to allow for proper and necessary flexing of the surgical implant 212 during flexing of the joint 16 into which the surgical implant 212 is inserted.

Additionally, as shown in FIG. 2A, the first grommet 214A can be positioned substantially about a portion of the proximal stem 224 substantially adjacent to the proximal hinge buttress 230. Somewhat similarly, the second grommet 214B can be positioned substantially about a portion of the distal stem 226 substantially adjacent to the distal hinge buttress 232. Alternatively, the first grommet 214A can be positioned somewhat spaced apart from the proximal hinge buttress 230 and/or the second grommet 214B can be positioned somewhat spaced apart from the distal hinge buttress 232.

Further, in certain embodiments, the grommets 214A, 214B have a substantially cylindrical outer surface 238 that tapers slightly along with the taper of the corresponding stem 224, 226 as the distance from the corresponding hinge buttress 230, 232 increases. This tapering of the outer surface 238 is designed to facilitate surgical preparation. Stated in another manner, the outer surface 238 of the grommets 214A, 214B has a substantially circular cross-section that tapers slightly as the distance from the hinge 228 increases, such that the grommets 214A, 214B are shaped as a portion of a cone, i.e. a frustum of a cone.

Additionally, in some embodiments, the grommets 214A, 214B can include one or more anti-rotation features 240, e.g., anti-rotation ribs or anti-rotation grooves, along the outer surface 238 of the grommet 214A, 214B so as to inhibit relative rotation between the grommet 214A, 214B and the bone into which the surgical implant 212 and the grommet 214A, 214B is inserted. Moreover, the design of the grommets 214A, 214B enables the use of easy-to-use cannulated instrumentation for preparation of the bone for insertion of the surgical implant 212 with grommets 214A, 214B. For example, no broaching is necessary because the outer surface 238 of the grommets 214A, 214B is cone-shaped, which facilitates the use of a drill rather than broach. Further, as provided herein, cannulated tapered drill bits can be used to prepare the bone as necessary over a wire.

FIG. 2B is a side view of the surgical implant 212 including the pair of grommets 214A, 214B of FIG. 2A. In particular, FIG. 2B illustrates certain additional details regarding the design of the surgical implant 212. For example, in this embodiment, the proximal stem 224 is angled slightly in the upward direction relative to the distal stem 226 to correspond with the relative angling of the bones, e.g., the metatarsal 18 and the proximal phalanx 20 illustrated in FIG. 1A, when the foot 10 (illustrated in FIG. 1A) is in a relaxed position.

Additionally, as shown in FIG. 2B, the hinge center section 234 includes an upper portion 242 and a lower portion 244 that can be integrally formed with one another. Further, the upper portion 242 can be somewhat V-shaped, and the lower portion 244 can be somewhat inverted V-shaped. Moreover, in certain embodiments, the upper portion 242 is somewhat larger than the lower portion 244. Stated in another manner, the V-shape of the upper portion 242 is somewhat deeper than the inverted V-shape of the lower portion 244.

FIG. 2C is a top view of the surgical implant 212 including the pair of grommets 214A, 214B of FIG. 2A. More particularly, FIG. 2C illustrates still further details regarding the design of the surgical implant 212. For example, as shown in FIG. 2C, the hinge center section 234 can be somewhat wider directly adjacent to the proximal hinge buttress 230 than it is directly adjacent to the distal hinge buttress 232. Stated in another manner, the hinge center section 234 tapers inwardly slightly from the proximal hinge buttress 230 toward the distal hinge buttress 232.

Additionally, in this embodiment, the proximal stem 224 is somewhat larger, i.e. wider and longer, than the distal stem 226. Moreover, because the proximal stem 224 is larger than the distal stem 226, the first grommet 214A is also somewhat larger, i.e. larger diameter around the outer surface 238, than the second grommet 214B.

FIG. 2D is a sectional view of the surgical implant 212 including the pair of grommets 214A, 214B taken on line D-D in FIG. 2B. It should be noted that neither the proximal stem 224 nor the first grommet 214A are visible in FIG. 2D.

In some embodiments, as shown in FIG. 2D, the grommets 214A, 214B have an internal shape, i.e. an inner surface 246, that matches the shape of an outer surface 248 of the corresponding stem 224, 226. Additionally, the shape of the inner surface 246 of the grommets 214A, 214B and/or the shape of the outer surface 248 of the stems 224, 226 can include anti-rotation features such that relative rotation between the stems 224, 226 and the grommets 214A, 214B is effectively inhibited. For example, in one embodiment, the inner surface 246 of the grommets 214A, 214B and/or the outer surface 248 of the stems 224, 226 can have a non-circular shape that does not allow for relative rotation between the stems 224, 226 and the grommets 214A, 214B. Additionally and/or alternatively, the stems 224, 226 and the grommets 214A, 214B can include specific corresponding features, e.g., ribs and grooves, that are designed to engage one another so as to inhibit the relative rotation between the stems 224, 226 and the grommets 214A, 214B.

FIG. 3 is a perspective view of another embodiment of a surgical implant 312 including a pair of grommets 314A, 314B having features of the present invention included therewith. As illustrated, the surgical implant 312 and the grommets 314A, 314B are substantially similar to the surgical implant 212 and the grommets 214A, 214B illustrated and described above in relation to FIGS. 2A-2D. For example, in this embodiment, the surgical implant 312 again includes a proximal stem 324, a distal stem 326, and a hinge 328 that are substantially similar to the proximal stem 224, the distal stem 226, and the hinge 228 illustrated and described above in relation to FIGS. 2A-2D.

Additionally, similar to the previous embodiment, the surgical implant 312 again includes the first grommet 314A that is positioned substantially about a portion of the proximal stem 324 substantially adjacent to a proximal hinge buttress 330, and the second grommet 314B that is positioned substantially about a portion of the distal stem 326 substantially adjacent to a distal hinge buttress 332. However, in this embodiment, the grommets 314A, 314B are integrally formed with the proximal stem 324 and the distal stem 326, respectively. With this design, although flexing of the foot 10 (illustrated in FIG. 1A) may result in some relative movement between the grommets 314A, 314B and the bones into which the surgical implant is implanted, the interface between the grommets 314A, 314B and the bones will be much less than may otherwise exist between the stems 324, 326 and the bones.

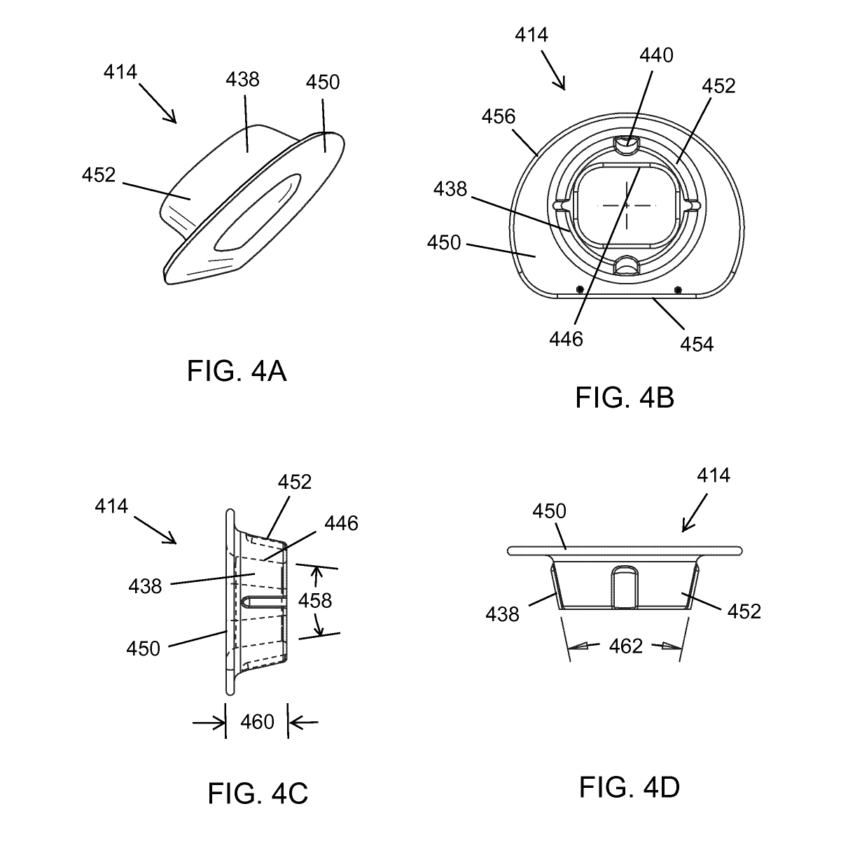

FIG. 4A is a perspective view of an embodiment of a grommet 414 having features of the present invention. In particular, FIG. 4A illustrates a perspective view of the grommet 414 including various features as described in detail herein above. For example, in one embodiment, the grommet 414 illustrated in FIG. 4A is sized and shaped such that it can be positioned about the distal stem 226 (illustrated, for example, in FIG. 2A) prior to insertion of the surgical implant 212 (illustrated, for example, in FIG. 2A). Stated in another manner, in such embodiment, the grommet 414 can be used as the second grommet 14B illustrated in FIG. 1A. Alternatively, the grommet 414 can be used for other suitable purposes.

The design of the grommet 414 can be varied to suit the specific design requirements of the surgical implant 212. In the embodiment illustrated in FIG. 4A, the grommet 414 includes a base 450 and a sleeve 452.

As illustrated, the base 450 can be a flat, ring-like member that fits around the distal stem 226 of the surgical implant 212. More specifically, the base 450 can be sized and shaped to be positioned around the distal stem 226 substantially adjacent to the hinge 228 (illustrated in FIG. 2A). In one embodiment, the base 450 can be positioned around the distal stem 226 substantially adjacent to the distal hinge buttress 232 (illustrated in FIG. 2A). Alternatively, the grommet 414 can be positioned relative to the surgical implant 212 in a different manner. For example, the grommet 414 can be positioned around the distal stem 226 spaced apart from the hinge 228.

Additionally, the sleeve 452 can be fixedly secured to and/or integrally formed with the base 450. Further, as illustrated, the sleeve 452 extends away from the base 450. In one embodiment, the sleeve 452 can be sized and shaped to be positioned around the distal stem 226 of the surgical implant 212. Moreover, the sleeve 452 can have an outer surface 438 that is sized and shaped to fit securely within the medullary canal in a bone, e.g., within the medullary canal in the proximal phalanx 20 (illustrated in FIG. 1A). With this design, the grommet 414 can remain substantially stationary relative to the proximal phalanx 20 during any flexing of the foot 10 (illustrated in FIG. 1A). Additionally, the grommet 414 can thus minimize and/or inhibit any direct interface between the distal stem 226 and the proximal phalanx 20 that may otherwise cause discomfort for the patient.

Further, in certain embodiments, the grommet 414 can be made from titanium, stainless steel, polyether ether ketone (PEEK), a cobalt-chromium alloy, or another suitable material.

FIG. 4B is an end view of the grommet 414 of FIG. 4A. In particular, FIG. 4B illustrates certain additional details and features of the grommet 414 illustrated in FIG. 4A. For example, in one embodiment, FIG. 4B illustrates that the base 450 of the grommet 414 can include a substantially flat bottom 454 and a rounded upper portion 456. In such embodiment, when the surgical implant 212 (illustrated in FIG. 2A) is implanted in the patient, e.g., in the foot 10 (illustrated in FIG. 1A) of the patient, the bottom 454 of the base 450 of the grommet 414 will be positioned below the distal stem 226 (illustrated in FIG. 2A) and toward the bottom of the foot 10. Alternatively, the base 450 can have a different design than that illustrated in FIG. 4B. For example, in one embodiment, the base 450 can be substantially completely circle-shaped.

Additionally, FIG. 4B further illustrates the general shape of the outer surface 438 of the sleeve 452 of the grommet 414. In particular, as illustrated in this embodiment, the outer surface 438 of the sleeve 452 is substantially cylinder-shaped or frustum-shaped. Moreover, one or more anti-rotation features 440 can be positioned about the outer surface 438 of the sleeve 452 so as to inhibit relative rotation between the grommet 414 and the bone, e.g., the proximal phalanx 20 (illustrated in FIG. 1A), into which the surgical implant 212 and the grommet 414 are inserted. For example, the anti-rotation features 440 can include one or more anti-rotation ribs that extend away from the rest of the outer surface 438 of the sleeve 452 and/or one or more anti-rotation grooves that are formed into the outer surface 438 of the sleeve 452. Alternatively, the anti-rotation features 440 can have a different design. Still alternatively, the outer surface 438 of the sleeve 452 can be another suitable design or shape.

Further, FIG. 4B also illustrates the general shape of the inner surface 446 of the grommet 414, i.e. the inner surface 446 of the base 450 and/or the sleeve 452 of the grommet 414. The shape of the inner surface 446 can be designed to match the shape of the outer surface of the distal stem 226. As illustrated in this embodiment, the inner surface 446 can have a non-circular shape that inhibits any relative rotation between the grommet 414 and the outer surface of the distal stem 226. For example, the inner surface 446 can be substantially rectangle-shaped, with rounded corners. Alternatively, the inner surface 446 can have another suitable non-circular shape, e.g., square, oval, triangle, hexagon, or other suitable shape.

FIG. 4C is a side view of the grommet 414 of FIG. 4A. In particular, FIG. 4C also illustrates certain additional details and features of the grommet 414 illustrated in FIG. 4A. For example, FIG. 4C illustrates that the outer surface 438 of the sleeve 452 tapers slightly as the sleeve 452 extends away from the base 450 of the grommet 414. As noted above, the taper of the outer surface 438 generally corresponds to the taper of the corresponding stem, e.g., the distal stem 226 illustrated in FIG. 2A, about which the grommet 414 is positioned.

Additionally, FIG. 4C further illustrates an inner taper angle 458 of the inner surface 446 (illustrated in phantom) of the sleeve 452 as the sleeve 452 extends away from the base 450. The inner taper angle 458 largely follows the taper of the corresponding stem, e.g., the distal stem 226 illustrated in FIG. 2A, about which the grommet 414 is positioned. In certain embodiments, the inner taper angle 458 can be between approximately 13.0 and 18.0 degrees. For example, in some non-exclusive alternative embodiments, the inner taper angle 458 can be approximately, 13.0, 13.5, 14.0, 14.5, 15.0, 15.5, 16.0, 16.5, 17.0, 17.5 or 18.0 degrees. Alternatively, the inner taper angle 458 can be greater than 18.0 degrees, less than 13.0 degrees, or some other value between 13.0 and 18.0 degrees.

Moreover, FIG. 4C illustrates a depth 460 of the grommet 414 from the base 450 to the end of the sleeve 452 away from the base 450. In certain embodiments, the depth 460 of the grommet 414 can be between approximately 0.10 inches and 0.20 inches. For example, in certain non-exclusive alternative embodiments, the depth 460 of the grommet 414 can be approximately 0.10, 0.125, 0.15, 0.175 or 0.20 inches. Alternatively, the depth 460 can be greater than 0.20 inches, less than 0.10 inches, or some other value between 0.10 and 0.20 inches.

FIG. 4D is a top view of the grommet 414 of FIG. 4A. More specifically, FIG. 4D illustrates an outer taper angle 462 of the outer surface 438 of the sleeve 452 as the sleeve 452 extends away from the base 450. In certain embodiments, the outer taper angle 462 can be between approximately 20.0 and 30.0 degrees. For example, in some non-exclusive alternative embodiments, the outer taper angle 462 can be approximately, 20.0, 22.0, 24.0, 26.0, 28.0 or 30.0 degrees. Alternatively, the outer taper angle 462 can be greater than 30.0 degrees, less than 20.0 degrees, or some other value between 20.0 and 30.0 degrees.

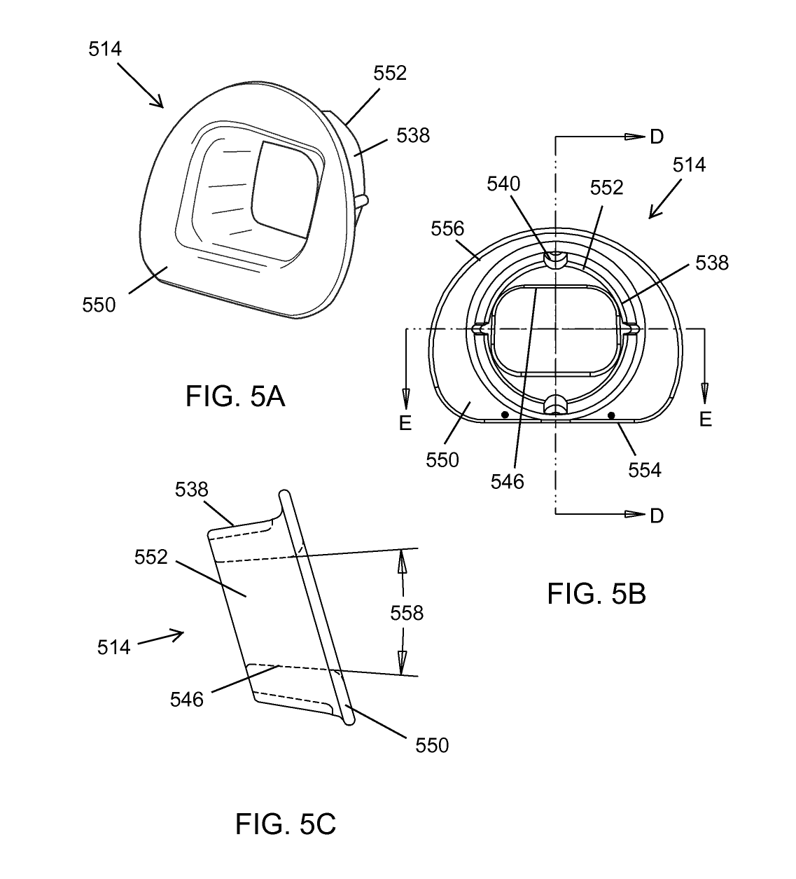

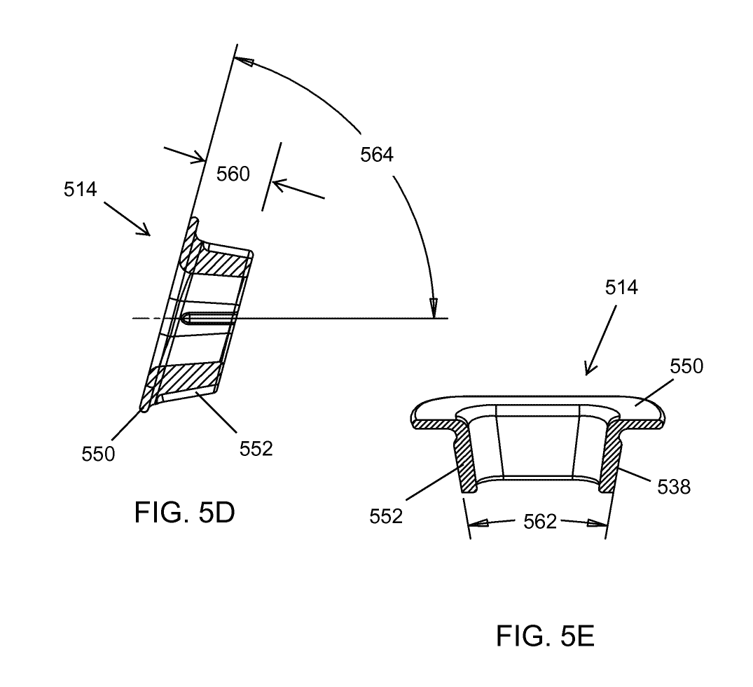

FIG. 5A is a perspective view of another embodiment of a grommet 514 having features of the present invention. In particular, FIG. 5A illustrates a perspective view of the grommet 514 including various features as described in detail herein above. For example, in one embodiment, the grommet 514 illustrated in FIG. 5A is sized and shaped such that it can be positioned about the proximal stem 224 (illustrated, for example, in FIG. 2A) prior to insertion of the surgical implant 212 (illustrated, for example, in FIG. 2A). Stated in another manner, in such embodiment, the grommet 514 can be used as the first grommet 14A illustrated in FIG. 1A. Alternatively, the grommet 514 can be used for other suitable purposes.

The grommet 514 illustrated in FIG. 5A is somewhat similar to the grommet 414 illustrated and described in relation to FIGS. 4A-4D. For example, the grommet 514 includes a base 550 and a sleeve 552 that are somewhat similar to the base 450 and the sleeve 452 illustrated and described above.

In this embodiment, the base 550 can be a flat, ring-like member that fits around the proximal stem 224 of the surgical implant 212. More specifically, the base 550 can be sized and shaped to be positioned around the proximal stem 224 substantially adjacent to the hinge 228 (illustrated in FIG. 2A). In one embodiment, the base 550 can be positioned around the proximal stem 224 substantially adjacent to the proximal hinge buttress 230 (illustrated in FIG. 2A). Alternatively, the grommet 514 can be positioned relative to the surgical implant 212 in a different manner. For example, the grommet 514 can be positioned around the proximal stem 224 spaced apart from the hinge 228.

Additionally, the sleeve 552 can be fixedly secured to and/or integrally formed with the base 550. Further, the sleeve 552 extends away from the base 550. In one embodiment, the sleeve 552 can be sized and shaped to be positioned around the proximal stem 224 of the surgical implant 212. Moreover, the sleeve 552 can have an outer surface 538 that is sized and shaped to fit securely within the medullary canal in a bone, e.g., within the medullary canal in the metatarsal 18 (illustrated in FIG. 1A). With this design, the grommet 514 can remain substantially stationary relative to the metatarsal 18 during any flexing of the foot 10 (illustrated in FIG. 1A). Additionally, the grommet 514 can thus minimize and/or inhibit any direct interface between the proximal stem 224 and the metatarsal 18 that may otherwise cause discomfort for the patient.

FIG. 5B is an end view of the grommet 514 of FIG. 5A. In particular, FIG. 5B illustrates certain additional details and features of the grommet 514 illustrated in FIG. 5A. Similar to the previous embodiment, the base 550 of the grommet 514 can again include a substantially flat bottom 554 and a rounded upper portion 556. Alternatively, the base 550 can have a different design than that illustrated in FIG. 5B. For example, in one embodiment, the base 550 can be substantially completely circle-shaped.

Additionally, FIG. 5B further illustrates the general shape of the outer surface 538 of the sleeve 552 of the grommet 514. For example, in one embodiment, the outer surface 538 of the sleeve 552 can be substantially cylinder-shaped or frustum-shaped, and can include one or more anti-rotation features 540 positioned about the outer surface 538 so as to inhibit relative rotation between the grommet 514 and the bone, e.g., the metatarsal 18 (illustrated in FIG. 1A), into which the surgical implant 212 and the grommet 514 are inserted. In certain embodiments, the anti-rotation features 540 can include one or more anti-rotation ribs that extend away from the rest of the outer surface 538 of the sleeve 552 and/or one or more anti-rotation grooves that are formed into the outer surface 538 of the sleeve 552. Alternatively, the anti-rotation features 540 can have a different design. Still alternatively, the outer surface 538 of the sleeve 552 can be another suitable design or shape.

Further, FIG. 5B also illustrates that the inner surface 546 of the grommet 514, i.e. the inner surface 546 of the base 550 and/or the sleeve 552 of the grommet 514 can again have a non-circular shape that inhibits any relative rotation between the grommet 514 and the outer surface of the proximal stem 224. Moreover, the shape of the inner surface 546 can be designed to match the shape of the outer surface of the proximal stem 224. For example, the inner surface 546 can be substantially rectangle-shaped, with rounded corners. Alternatively, the inner surface 546 can have another suitable non-circular shape, e.g., square, oval, triangle, hexagon, or other suitable shape.

FIG. 5C is a side view of the grommet 514 of FIG. 5A. In this embodiment, the outer surface 538 of the sleeve 552 again tapers slightly as the sleeve 552 extends away from the base 550 of the grommet 514. Additionally, FIG. 5C further illustrates an inner taper angle 558 of the inner surface 546 (illustrated in phantom) of the sleeve 552 as the sleeve 552 extends away from the base 550. The inner taper angle 558 largely follows the taper of the corresponding stem, e.g., the proximal stem 224 illustrated in FIG. 2A, about which the grommet 514 is positioned. In certain embodiments, the inner taper angle 558 can be between approximately 6.0 and 11.0 degrees. For example, in some non-exclusive alternative embodiments, the inner taper angle 558 can be approximately, 6.0, 6.5, 7.0, 7.5, 8.0, 8.5, 9.0, 9.5, 10.0, 10.5 or 11.0 degrees. Alternatively, the inner taper angle 558 can be greater than 11.0 degrees, less than 6.0 degrees, or some other value between 6.0 and 11.0 degrees.

FIG. 5D is a sectional view of the grommet 514 taken on line D-D in FIG. 5B. In particular, FIG. 5D illustrates certain additional details and features of the grommet 514 illustrated in FIG. 5A. For example, FIG. 5D illustrates a depth 560 of the grommet 514 from the base 550 to the end of the sleeve 552 away from the base 550. In certain embodiments, the depth 560 of the grommet 514 can be between approximately 0.12 inches and 0.22 inches. For example, in certain non-exclusive alternative embodiments, the depth 560 of the grommet 514 can be approximately 0.12, 0.14, 0.16, 0.18, 0.20 or 0.22 inches. Alternatively, the depth 560 can be greater than 0.22 inches, less than 0.12 inches, or some other value between 0.12 and 0.22 inches.

Additionally, FIG. 5D further illustrates that the grommet 514 has grommet angle 564 that is designed to correspond to the angle of the proximal stem 224 (illustrated in FIG. 2A) relative to the distal stem 226 (illustrated in FIG. 2A). More particularly, as noted above, in certain embodiments, the proximal stem 224 is angled slightly in the upward direction relative to the distal stem 226 to correspond with the relative angling of the bones, e.g., the metatarsal 18 and the proximal phalanx 20 illustrated in FIG. 1A, when the foot 10 (illustrated in FIG. 1A) is in a relaxed position. In such embodiments, the grommet angle 564 can be designed to correspond to relative angle between the proximal stem 224 and the distal stem 226. In some embodiments, the grommet angle 564 can be between approximately 70.0 and 80.0 degrees. For example, in certain non-exclusive alternative embodiments, the grommet angle 564 can be approximately, 70.0, 72.0, 74.0, 75.0, 76.0, 78.0, or 80.0 degrees. Alternatively, the grommet angle 564 can be greater than 80.0 degrees, less than 70.0 degrees, or some other value between 70.0 and 80.0 degrees.

FIG. 5E is another sectional view of the grommet 514 taken on line E-E in FIG. 5B. In particular, FIG. 5E illustrates an outer taper angle 562 of the outer surface 538 of the sleeve 552 as the sleeve 552 extends away from the base 550. In certain embodiments, the outer taper angle 562 can be between approximately 15.0 and 25.0 degrees. For example, in some non-exclusive alternative embodiments, the outer taper angle 562 can be approximately, 15.0, 17.0, 19.0, 20.0, 21.0, 23.0 or 25.0 degrees. Alternatively, the outer taper angle 562 can be greater than 25.0 degrees, less than 15.0 degrees, or some other value between 15.0 and 25.0 degrees.

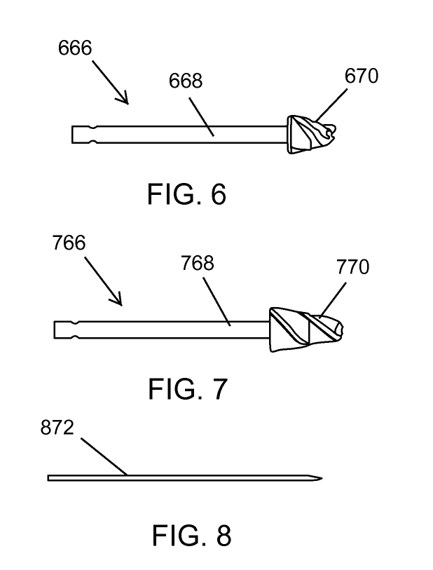

FIG. 6 is a simplified side view of an embodiment of a cannulated tapered drill bit 666 that can be used with the present invention. In particular, FIG. 6 illustrates a drill bit 666 that can be used to prepare the bone, e.g., the metatarsal 18 (illustrated in FIG. 1A) and/or the proximal phalanx 20 (illustrated in FIG. 1A), as necessary for insertion of a surgical implant, e.g., the surgical implant 212 illustrated in FIG. 2A. Additionally, the drill bit 666 can be used to prepare the bone 18, 20 for such a surgical implant 212 that includes a pair of grommets 214A, 214B (illustrated in FIG. 2A) provided therewith.

As illustrated in this embodiment, the drill bit 666 includes a bit shaft 668, and a bit head 670 that is secured to and/or integrally formed with the bit shaft 668. The bit shaft 668 can be substantially cylindrical in shape and can be selectively positioned within a drill (not illustrated) during use. Additionally, the bit head 670 can be sized and shaped as necessary to properly prepare the bone 18, 20 to receive the surgical implant 212.

FIG. 7 is a simplified side view of another embodiment of a cannulated tapered drill bit 766 that can be used with the present invention. In particular, similar to the previous embodiment, FIG. 7 illustrates a drill bit 766 that can be used to prepare the bone, e.g., the metatarsal 18 (illustrated in FIG. 1A) and/or the proximal phalanx 20 (illustrated in FIG. 1A), as necessary for insertion of a surgical implant, e.g., the surgical implant 212 illustrated in FIG. 2A. Additionally, the drill bit 766 can be used to prepare the bone 18, 20 for such a surgical implant 212 that includes a pair of grommets 214A, 214B (illustrated in FIG. 2A) provided therewith.

The design of the drill bit 766 is somewhat similar to the drill bit 666 illustrated and described in relation to FIG. 6. For example, the drill bit 766 again includes a bit shaft 768, and a bit head 770 that is secured to and/or integrally formed with the bit shaft 768. The bit shaft 768 can be substantially cylindrical in shape and can be selectively positioned within a drill (not illustrated) during use. Additionally, the bit head 770 can be sized and shaped as necessary to properly prepare the bone 18, 20 to receive the surgical implant 212. As illustrated in this embodiment, the bit head 770 is somewhat larger, i.e. wider and/or longer, than the bit head 670 in the previous embodiment.

FIG. 8 is a simplified side view of a wire 872 that can be used with the drill bits 666, 766 of FIGS. 6 and 7. In particular, the wire 344 can be used to guide the drill bits 666, 766 during preparation of the bone, e.g., the metatarsal 18 (illustrated in FIG. 1A) and/or the proximal phalanx 20 (illustrated in FIG. 1A), for insertion of the surgical implant 212 (illustrated in FIG. 2A).

While a number of exemplary aspects and embodiments of a grommet 14A, 14B for use with a surgical implant 12 have been shown and disclosed herein above, those of skill in the art will recognize certain modifications, permutations, additions and sub-combinations thereof. It is therefore intended that the grommet 14A, 14B shall be interpreted to include all such modifications, permutations, additions and sub-combinations as are within their true spirit and scope, and no limitations are intended to the details of construction or design herein shown.

What is claimed is:

1. A grommet for use with a surgical implant, the grommet comprising:
a base that can be positioned around a portion of the surgical implant;
a sleeve that is connected to the base and extends away from the base, the sleeve including an inner surface having a non-circular shape, and an outer surface that is substantially cylinder-shaped.

2. The grommet of claim 1 wherein the outer surface of the sleeve tapers as the sleeve extends away from the base.

3. The grommet of claim 2 wherein the outer surface of the sleeve has a taper angle of between approximately 20.0 and 25.0 degrees.

4. The grommet of claim 1 wherein the sleeve includes one or more anti-rotation features that are positioned along the outer surface of the sleeve.

5. The grommet of claim 4 wherein the anti-rotation features include one or more anti-rotation ribs that extend away from the outer surface of the sleeve.

6. The grommet of claim 4 wherein the anti-rotation features include one or more anti-rotation grooves that are formed into the outer surface of the sleeve.

7. The grommet of claim 1 wherein the base is inhibited from rotating relative to the portion of the surgical implant.

8. The grommet of claim 1 wherein the surgical implant includes a stem, and wherein the grommet can be positioned around a portion of the stem.

9. The grommet of claim 8 wherein the stem has an outer surface having a first shape, and wherein the inner surface of the sleeve has a second shape, the second shape being substantially similar to the first shape.

10. The grommet of claim 9 wherein the second shape is non-circular such that the grommet is inhibited from rotating relative to the stem.

11. A surgical implant for implanting into bone material, the surgical implant including a stem and the grommet of claim 1 that is positioned around the stem.

12. The surgical implant of claim 11 wherein the sleeve includes one or more anti-rotation features that are positioned along the outer surface of the sleeve, the anti-rotation features inhibiting the grommet from rotating relative to the bone material.

13. A grommet for use with a surgical implant that can be implanted into bone material, the grommet comprising:
a base that can be positioned around a portion of the surgical implant; and
a sleeve that is connected to the base and extends away from the base, the sleeve including an outer surface that is substantially cylinder-shaped, and one or more anti-rotation features that are positioned along the outer surface of the sleeve.

14. The grommet of claim 13 wherein the surgical implant includes a stem, and wherein the grommet can be positioned around a portion of the stem.

15. The grommet of claim 14 wherein the stem has an outer surface having a first shape, and wherein the sleeve has an inner surface having a second shape, the second shape being substantially similar to the first shape.

16. The grommet of claim 15 wherein the second shape is non-circular such that the grommet is inhibited from rotating relative to the stem.

17. The grommet of claim 13 wherein the outer surface of the sleeve tapers as the sleeve extends away from the base, the outer surface of the sleeve having a taper angle of between approximately 20.0 and 25.0 degrees.

18. A surgical implant for implanting into bone material, the surgical implant including a stem and the grommet of claim 13 that is positioned around the stem.

19. The surgical implant of claim 18 wherein the anti-rotation features inhibit the grommet from rotating relative to the bone material.

20. A grommet for use with a surgical implant that can be implanted into bone material, the surgical implant including a stem, the grommet comprising:
a base that can be positioned around a portion of the stem; and
a sleeve that is connected to the base and extends away from the base, the sleeve including (i) an inner surface having a non-circular shape such that the grommet is inhibited from rotating relative to the stem, (ii) an outer surface that is substantially cylinder-shaped, and (iii) one or more anti-rotation features that are positioned along the outer surface of the sleeve, wherein when the surgical implant is implanted into the bone material, the one or more anti-rotation features inhibit the grommet from rotating relative to the bone material.
